(19) United States
(12) Patent Application Publication
(10) Pub. No.: US 2023/0352161 A1
(43) Pub. Date: Nov. 2, 2023

(54) MEDICAL DEVICE MANAGEMENT SYSTEM, MANAGEMENT SERVER, MEDICAL DEVICE, AND MEDICAL DEVICE MANAGEMENT METHOD

(71) Applicant: OLYMPUS CORPORATION, Tokyo (JP)

(72) Inventors: Haruna TADAKOSHI, Tokyo (JP); Koki TOKUNAGA, Tokyo (JP); Yuya ISHIDA, Tokyo (JP); Takeo SUZUKI, Tokyo (JP)

(73) Assignee: OLYMPUS CORPORATION, Tokyo (JP)

(21) Appl. No.: 18/220,937

(22) Filed: Jul. 12, 2023

Related U.S. Application Data
(63) Continuation of application No. PCT/JP2021/024030, filed on Jun. 24, 2021.

(51) Int. Cl. G16H 40/40; G16H 40/67

(52) U.S. Cl. CPC G16H 40/40; G16H 40/67

(57) ABSTRACT

A first acquisition unit acquires first identification information for identifying a first medical device having a first constituent component and a second constituent component. A second acquisition unit acquires the status of the first constituent component incorporated in a second medical device or second identification information for identifying the constituent component incorporated in the second medical device based on the first identification information. A recording unit records the status of the first constituent component or the second identification information and third identification information for identifying the second medical device in association with each other.

### CROSS-REFERENCE TO RELATED APPLICATIONS

This application is based upon and claims the benefit of priority from the International Application No. PCT/JP2021/024030, filed on Jun. 24, 2021, the entire contents of which are incorporated herein by reference.

### BACKGROUND

### 1. Field of the Disclosure

The present disclosure relates to a technology for managing the status of components constituting a medical device.

### 2. Description of the Related Art

In order to ensure medical safety and infection prevention, there are medical instrument products that are designed to be used only once. Such products are called single-use medical devices, and once those devices are used in a medical facility, the devices are collected by specialized collectors.

The specification of U.S. patent Ser. No. 10/226,163 discloses a system for determining whether a single-use endoscope has been used or not. If the single-use endoscope has not been used, the system allows for the activation of the endoscope. On the other hand, if the endoscope has been used, the system presents a message to the user indicating that the use of the endoscope is not authorized and does not activate the endoscope.

In recent years, from the viewpoints of effective utilization of resources and reduction of medical waste, remanufacturing of used single-use medical devices has been attracting attention, in which the manufacturer/distributor is responsible for disassembling, cleaning, replacing parts, reassembling, sterilizing, etc., so that the devices can be used again.

### SUMMARY

There are cases where components that constitute single-use medical devices are intentionally set to have lower strength and durability compared to components that constitute reusable medical devices that can be used repeatedly. Therefore, the number of time the components are reused and the period of time in which the components are reused for medical operations are preferably limited. In this background, a purpose of the present disclosure is to provide a technology for managing the status of constituent components of a medical device.

A medical device management system according to one embodiment of the present disclosure includes: a first acquisition unit that acquires first identification information for identifying a first medical device having a first constituent component and a second constituent component; a second acquisition unit that acquires the status of the first constituent component incorporated in a second medical device or second identification information for identifying the first constituent component incorporated in the second medical device based on the first identification information; and a recording unit that records the status of the first constituent component or the second identification information and third identification information for identifying the second medical device in association with each other.

A management server according to another embodiment of the present disclosure is a management server that manages a medical device and includes: a first acquisition unit that acquires first identification information for identifying a first medical device having a first constituent component and a second constituent component; a second acquisition unit that acquires the status of the first constituent component incorporated in a second medical device or second identification information for identifying the first constituent component incorporated in the second medical device based on the first identification information; and a recording unit that records the status of the first constituent component or the second identification information and third identification information for identifying the second medical device in association with each other.

A medical device according to yet another embodiment of the present disclosure is a medical device having a plurality of constituent components, including: an acquisition unit that acquires the status of a constituent component that used to be incorporated in another medical device or identification information for identifying the constituent component that used to be incorporated in the other medical device; and a recording unit that records the status of the constituent component or the identification information and identification information for identifying the medical device in association with each other.

A medical device management method according to still another embodiment of the present disclosure includes: acquiring first identification information for identifying a first medical device having a first constituent component and a second constituent component; acquiring the status of the first constituent component incorporated in a second medical device or second identification information for identifying the first constituent component incorporated in the second medical device based on the first identification information; and recording the status of the first constituent component or the second identification information and third identification information for identifying the second medical device in association with each other.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the configuration of a medical device management system;

FIG. 2 is a diagram showing functional blocks of a management server;

FIG. 3 is a diagram showing an example of an external view of an endoscope;

FIG. 4 is a diagram showing an example of various components constituting the endoscope;

FIG. 5 is a diagram showing examples of an upper limit usage count and an upper limit time of use;

FIG. 6 is a diagram showing examples of the use status of constituent components of a manufactured endoscope;

FIG. 7 is a diagram showing examples of the use status of constituent components of a used endoscope;

FIG. 8 is a flowchart for managing the status of constituent components;

FIG. 9 is a diagram showing examples of the use status of constituent components of a manufactured endoscope;

FIG. 10 is a diagram showing different examples of the use status of constituent components of a used endoscope;

FIG. 11 is a diagram showing different examples of the use status of constituent components of a used endoscope;

FIG. 12 is a diagram showing examples of the use status of constituent components of a used endoscope;

FIG. 13 is a diagram showing examples of the use status of constituent components of a used endoscope;

FIG. 14 is a flowchart for managing the status of constituent components;

FIG. 15 is a diagram showing examples of records of constituent components of a manufactured endoscope;

FIG. 16 is a diagram showing examples of upper limit usage counts and upper limit times of use of constituent components set for a plurality of classes;

FIG. 17 is a diagram showing examples of upper limit transportation counts and upper limit transportation times set for constituent components;

FIG. 18 is a diagram showing examples of the transportation status of constituent components of a used endoscope;

FIG. 19 is a diagram showing a state where component IDs of a plurality of constituent components are associated with a scope ID; and

FIG. 20 is a diagram showing a state where component IDs of a plurality of constituent components are associated with a component ID of a specific constituent component.

### DETAILED DESCRIPTION

The disclosure will now be described by reference to the preferred embodiments. This does not intend to limit the scope of the present disclosure, but to exemplify the disclosure.

The present disclosure relates to a technology for managing the status of components constituting a medical device. In embodiments shown below, medical devices are single-use endoscopes. Alternatively, the medical devices may be single-use medical devices other than endoscopes. At medical facilities such as hospitals, single-use endoscopes are disposed of in disposal bins after use for examinations. Specialized collection companies collect the used single-use endoscopes from the medical facilities and transport the endoscopes to medical device manufacturers on a regular or as-needed basis. The medical device manufacturers disassemble the used single-use endoscopes, cleans the components, and remanufacture single-use endoscopes by combining multiple cleaned components.

Since the strength and durability of the components of a single-use endoscope differ for each component, the number of times the components can be used for endoscopic examinations and the time the components can be used need to be set properly for each component. Therefore, the medical device management system according to an embodiment has a function of determining for each constituent component of an endoscope whether or not the constituent component can be incorporated into a new single-use endoscope according to the past use and transportation results.

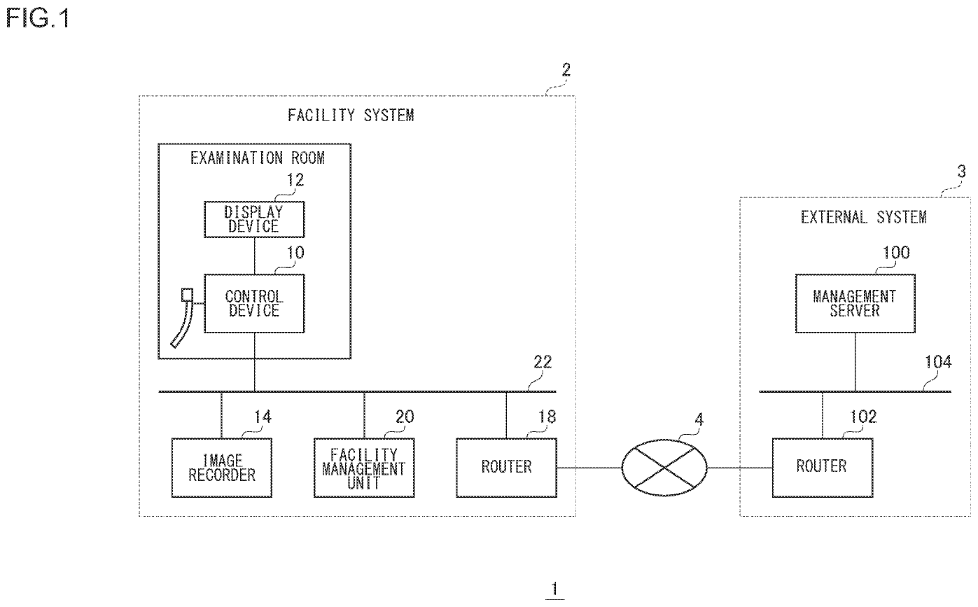

FIG. 1 shows the configuration of a medical device management system 1 according to the embodiment. The medical device management system 1 manages the status of the constituent components of a single-use endoscope (hereinafter often simply referred to as “endoscope”). The medical device management system 1 includes a facility system 2, which is installed inside a medical facility, and an external system 3, which is installed outside the medical facility. The facility system 2 and the external system 3 are communicably connected via Internet 4. The external system 3 may be operated by a medical device manufacturer that provides endoscopes to the medical facility and remanufactures new endoscopes by disassembling used endoscopes collected from the medical facility and taking out components.

The facility system 2 includes a control device 10, an image recorder 14, and a facility management unit 20. The control device 10, the image recorder 14, and the facility management unit 20 are communicably connected by a network 22 such as a local area network (LAN). The network 22 is connected to the Internet 4 via a router 18.

The control device 10 is provided in an examination room and connected to an endoscope, which is a medical device, so as to control the functions of the endoscope. When a medical worker operates an examination start button provided in the control device 10, the control device 10 displays an image of the inside of the patient's body being captured by the endoscope on the display unit 12, and the doctor observes each part of an organ that is displayed. When the doctor finds a lesion, the doctor operates a release switch of the endoscope so as to capture an image including the lesion. The control device 10 captures the image at the time when the release switch is operated and transmits the captured image to the image recorder 14. When observation of all organs is completed, the medical worker operates an examination end button provided on the control device 10 so as to finish the examination. The control device 10 may transmit a plurality of captured images all at once to the image recorder 14 after the examination is completed.

The image recorder 14 is an image server that records images captured during an endoscopic examination and accumulates images transmitted from the control device 10 in association with the identification information (examination ID) of the examination. As metadata, the date and time of the image capturing, identification information of the endoscope (hereinafter, also referred to as “scope ID”), information on the examination order, etc., may be added to a captured image.

The facility management unit 20 acquires information on the examination from the control device 10. When the control device 10 is connected to an endoscope, the control device 10 acquires the scope ID from the endoscope and transmits the scope ID along with the examination ID to the facility management unit 20. When the examination start button is operated, the control device 10 transmits the examination start information along with the examination ID to the facility management unit 20. When the examination end button is operated, the control device 10 transmits the examination end information along with the examination ID to the facility management unit 20.

When the examination start information includes the examination start time and the examination end information includes the examination end time, the facility management unit 20 extracts the examination start time and the examination end time and records the scope ID, the examination start time, and the examination end time in association with the examination ID. The facility management unit 20 may record the time when the examination start information is received as the examination start time and the time when the examination end information is received as the examination end time. The facility management unit 20 transmits the examination information including the examination ID, the scope ID, the examination start time, and the examination end time to the management server 100.

The external system 3 includes a management server 100 that manages the status of the components constituting a single-use endoscope. The management server 100 becomes connected to a network 104 such as a local area network (LAN) and becomes connected to the Internet 4 via a router 102. As described above, the external system 3 may be operated by a medical device manufacturer that provides endoscopes to the medical facility and reassembles endoscopes.

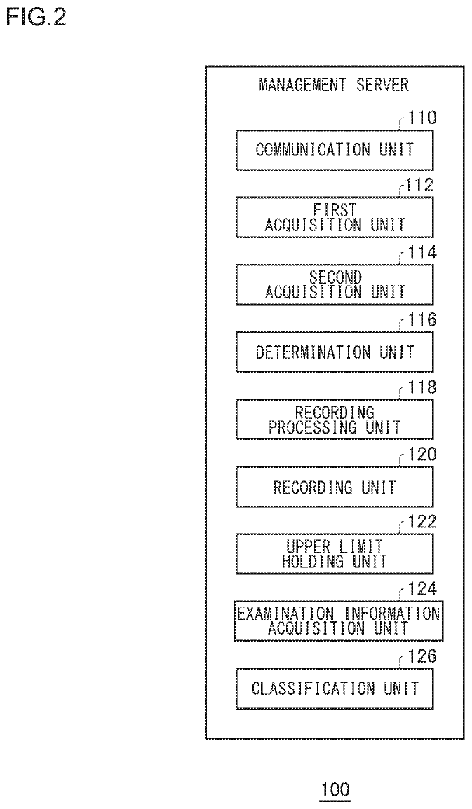

FIG. 2 shows functional blocks of the management server 100. The management server 100 manages single-use endoscopes and includes a communication unit 110, a first acquisition unit 112, a second acquisition unit 114, a determination unit 116, a recording processing unit 118, a recording unit 120, an upper limit holding unit 122, an examination information acquisition unit 124, and a classification unit 126. The configuration shown in FIG. 2 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

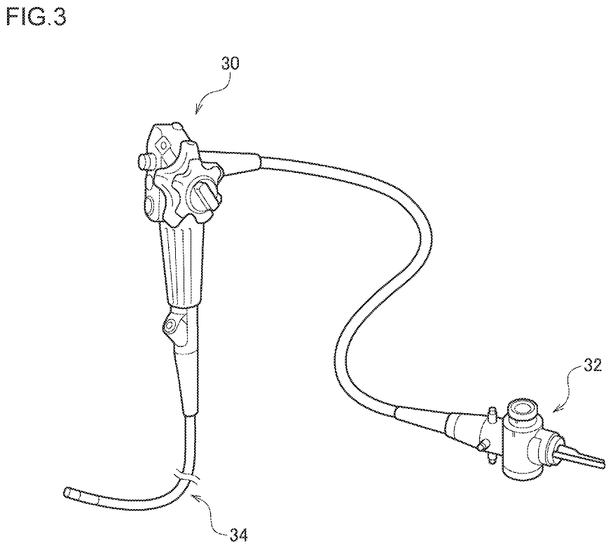

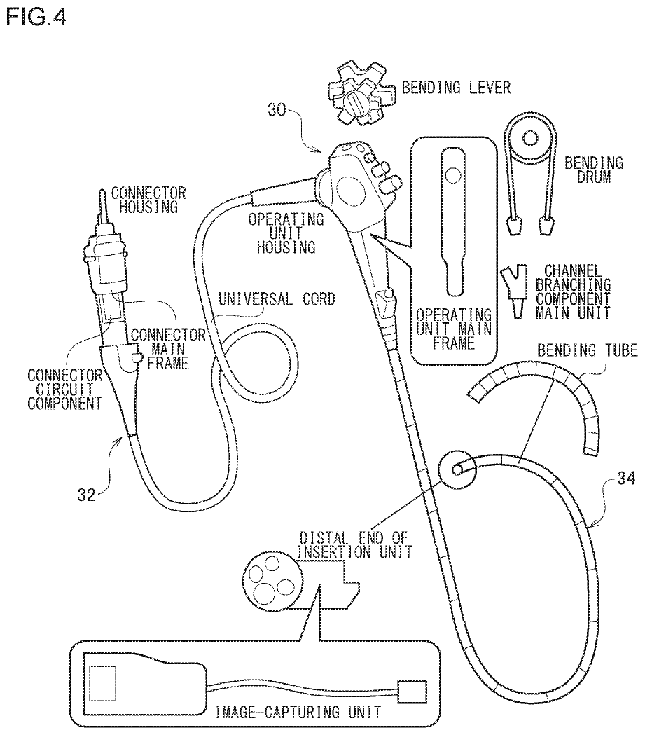

FIG. 3 shows an example of an external view of an endoscope. The endoscope has an operation unit 30 operated by a doctor, a connecting unit 32 connected to the control device 10, and an insertion unit 34 inserted into the patient's body. The operation unit 30, the connecting unit 32, and the insertion unit 34 are each composed of multiple components assembled together. FIG. 4 shows an example of various components constituting the endoscope. As shown in FIG. 4, the operation unit 30, the connecting unit 32, and the insertion unit 34 are each composed of various components.

There are cases where components that constitute single-use endoscopes are intentionally set to have lower strength and durability compared to components that constitute reusable endoscopes that can be used repeatedly. Therefore, the number of time the components are incorporated into a new medical device and used for medical operations and the period of time in which the components are used are preferably limited. Therefore, in the medical device management system 1 according to the embodiment, each constituent component of a single-use endoscope is set with an upper limit usage count for which the component can be used for an examination and an upper limit amount of time for which the component can be used for an examination.

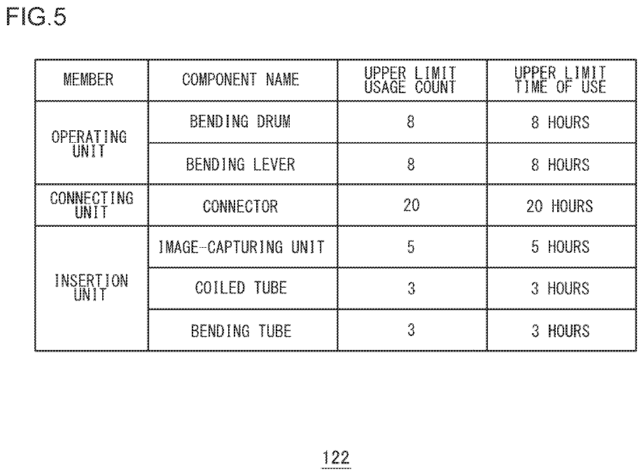

FIG. 5 shows examples of upper limit usage counts and upper limit times of use that are set for constituent components of a single-use endoscope. FIG. 5 representatively shows the upper limit usage counts and the upper limit times of use for six components constituting the single-use endoscope. Upper limit usage counts and upper limit times of use for other components may also be set.

The upper limit holding unit 122 holds the upper limit usage count and the upper limit time of use for each constituent component. The upper limit usage count is the maximum number of times a constituent component is used for an examination, in other words, the maximum number of times the constituent component is incorporated into an endoscope used for an examination. The upper limit time of use is the maximum amount of time used for an examination, in other words, the maximum total time an endoscope incorporating the constituent component is used for an examination. When the results of use of the constituent component exceeds either the upper limit usage count or the upper limit time of use, the constituent component is discarded. In the embodiment, the upper limit holding unit 122 holds the upper limit usage count and the upper limit time of use. Alternatively, the upper limit holding unit 122 may hold either one of the upper limit usage count and the upper limit time of use. The upper limit usage count and the upper limit time of use may be set by the medical device manufacturer in consideration of safety.

### First Embodiment

The recording unit 120 records the results of use of a constituent component in endoscopic examinations and manages the use status of the constituent component. In association with identification information (scope ID) of a manufactured endoscope, the recording unit 120 according to the first embodiment records the accumulated values of the usage count and the time of use for a constituent component constituting the endoscope. The usage count of the component is the number of times the endoscope incorporating the component has been used for examinations, and the time of use of the component is the total amount of time in which the endoscope incorporating the component has been used for examinations.

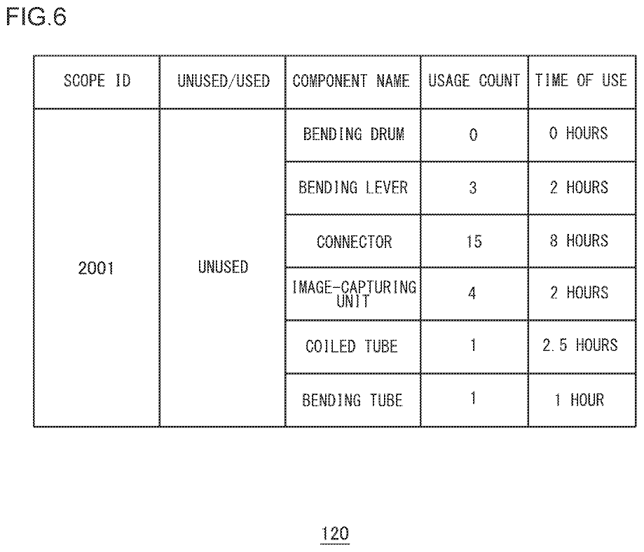

FIG. 6 shows examples of the use status of constituent components of a manufactured endoscope. The medical device manufacturer assigns a scope ID to a single-use endoscope manufactured by assembling components. In this example, scope ID “2001” is assigned to a manufactured endoscope, and the recording processing unit 118 records a component name, the usage count, and the time of use in the recording unit 120 in association with scope ID “2001”. The usage count and the time of use associated with each constituent component are the accumulated values to date and constitute the use status of the constituent component.

A bending drum (pulley for a bending operation) has a use status of zero for the usage count and zero for the time of use. The use status of zero times of use and zero hours of use indicates that the bending drum is new.

A bending lever (knob for a bending operation) has a use status of three for the usage count and two hours for the time of use. The three times of use in this case means that the lever for bending has been incorporated into three endoscopes and used in three examinations in the past, and the two hours of use means that the total time of the three examinations for which the lever has been used in the past is two hours.

A connector has a use status of 15 times of use and eight hours of use. As mentioned above, 15 times of use means that the connector has been incorporated into 15 endoscopes and used in 15 examinations in the past.

The recording processing unit 118 reads the latest use status of each constituent component from the recording unit 120 based on the scope ID of an endoscope in which the constituent component was most recently incorporated and creates a record associated with scope ID “2001”. A field for “unused/used” indicates whether the endoscope has not yet been used or has been used at the medical facility and is set to “unused” when the endoscope is manufactured.

Manufactured single-use endoscopes are packaged in sterile packs and transported from the manufacturing facility of the endoscopes to medical facilities in individual packaging boxes. At a medical facility, single-use endoscopes are stored in a storage while being kept in individual packaging boxes, and when it is time to be used in an endoscopic examination, the single-use endoscopes are taken out from the storage and brought to an examination room. Prior to the start of an examination, a medical worker opens a sterilization pack, takes out an endoscope, and connect the endoscope to the control device 10. In the following explanation, an endoscope with scope ID “2001” is connected to the control device 10.

When the control device 10 is connected to an endoscope, the control device 10 acquires scope ID “2001” from the endoscope and transmits scope ID “2001” along with the examination ID to the facility management unit 20. When the medical worker operates an examination start button, the control device 10 transmits examination start information along with the examination ID to the facility management unit 20. When the observation by a doctor is completed and the medical worker operates an examination end button, the control device 10 transmits examination end information along with the examination ID to the facility management unit 20. In the first embodiment, the facility management unit 20 acquires examination start time “2021/6/15 10:15” and examination end time “2021/6/15 11:15” and records scope ID “2001”, the examination start time “2021/6/15 10:15”, and the examination end time “2021/6/15 11:15” in association with the examination ID as examination information. After receiving the examination end information, the facility management unit 20 transmits the examination information including the examination ID, the scope ID, the examination start time, and the examination end time to the management server 100. The facility management unit 20 may transmit the examination information to the management server 100 after the end of work for the day at the medical facility.

In the management server 100, the communication unit 110 receives the examination information, and the examination information acquisition unit 124 acquires the examination information. The examination information acquisition unit 124 provides the acquired examination information to the recording processing unit 118. As described above, the examination information includes the examination ID, scope ID “2001”, the examination start time “2021/6/15 10:15”, and the examination end time “2021/6/15 11:15”. The recording processing unit 118 identifies the record of scope ID “2001” (see FIG. 6) in the recording unit 120.

The recording processing unit 118 updates the use status of the components constituting the endoscope based on the examination information. First, based on the examination start time “2021/6/15 10:15” and the examination end time “2021/6/15 11:15”, the recording processing unit 118 derives the time the examination was conducted (examination time) as one hour.

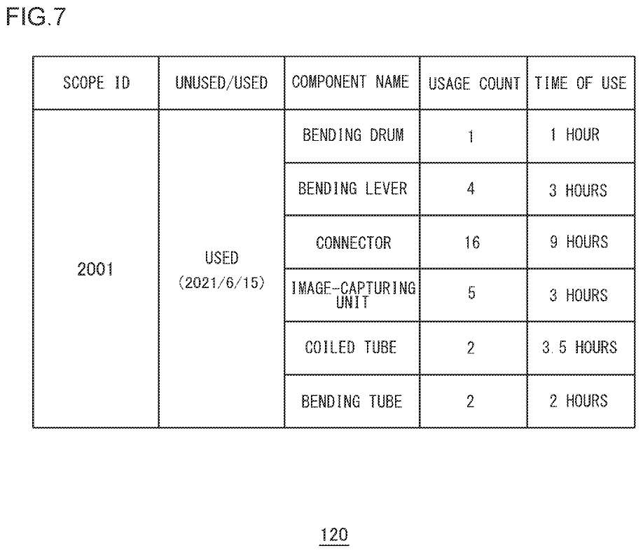

FIG. 7 shows examples of the use status of constituent components of a used endoscope. Compared to the use status shown in FIG. 6, the results of an examination performed on 2021/6/15 are reflected such that the usage count of each constituent component is incremented by “one” and the time of use is incremented by “one hour”. The recording processing unit 118 updates a record field and records the usage count and the time of use in the recording unit 120. When the record field is updated, the recording processing unit 118 changes the unused/used field to “used” and registers the date of use (examination date). Therefore, the latest status of the constituent components incorporated in the endoscope with scope ID “2001” is recorded in the recording unit 120 as of 2021/6/15.

Endoscopes used in medical facilities are collected by specialized collection companies and transported to the medical device manufacturer. The medical device manufacturer disassemble the used endoscopes and cleans the components. Based on the use status of each constituent component, the determination unit 116 determines whether or not the constituent component can be incorporated into a new endoscope, and constituent components that can be incorporated are sent to a manufacturing process.

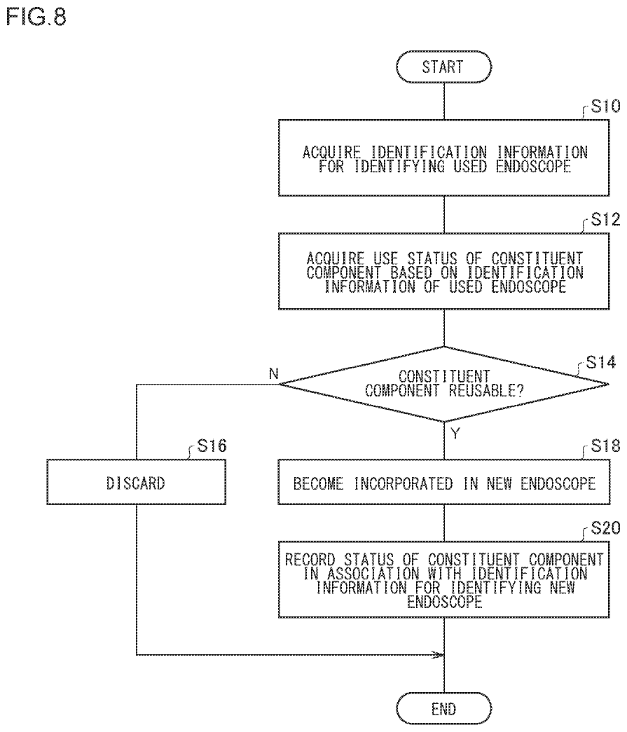

FIG. 8 is a flowchart for managing the status of constituent components. When a used endoscope is brought to a medical device manufacturer, the first acquisition unit 112 acquires identification information for identifying the used endoscope (S10). The first acquisition unit 112 acquires the scope ID of the used endoscope as the identification information. For example, if the connecting unit of the endoscope has an RF tag that records the scope ID, the first acquisition unit 112 may have a function of reading the scope ID from the RF tag. In this case, the first acquisition unit 112 acquires scope ID “2001”.

In the first embodiment, the constituent components that used to be incorporated in scope ID “2001” are scheduled to be incorporated into a specific new endoscope. The constituent components that used to be incorporated in scope ID “2001” may be scheduled to be incorporated into the same endoscope or may be scheduled to be incorporated into a different endoscope. In any case, the management server 100 may recognize an endoscope in which a constituent component that used to be incorporated in the endoscope with scope ID “2001” is to be newly incorporated. In other words, the management server 100 may recognize the scope ID of an endoscope in which each constituent component to be incorporated into a newly manufactured endoscope was most recently incorporated.

The second acquisition unit 114 acquires the use status of a constituent component to be incorporated into a new endoscope based on scope ID “2001” of the endoscope acquired by the first acquisition unit 112 (S12). More specifically, the second acquisition unit 114 refers to the recording unit 120 and acquires the use status of the constituent component scheduled to be incorporated into the new endoscope from a record including scope ID “2001” (see FIG. 7).

The determination unit 116 determines whether or not the constituent component can be incorporated into the new endoscope based on the status of the constituent component acquired by the second acquisition unit 114 (S14). More specifically, the determination unit 116 compares the usage count and the time of use of the constituent component recorded in the recording unit 120 with the upper limit usage count and the upper limit time of use held in the upper limit holding unit 122. The determination unit 116 determines that the constituent component cannot be incorporated into a new endoscope if the usage count included in the use status of the constituent component is equal to or greater than the upper limit usage count and determines that the constituent component cannot be incorporated into a new endoscope if the time of use included in the use status of the constituent component is equal to or greater than the upper limit time of use (N in S14). The constituent component determined to be non-reusable may be discarded (S16).

In the record shown in FIG. 7, the number of times an image-capturing unit has been used is five times, reaching the upper limit usage count of the image-capturing unit (see FIG. 5). Therefore, the determination unit 116 determines that the image-capturing unit has been used up to the upper limit usage count and that the image-capturing unit cannot be incorporated into a new endoscope.

In the record shown in FIG. 7, a coiled tube has been used for 3.5 hours, which exceeds the upper limit time of use of a coiled tube use of three hours (see FIG. 5). Therefore, the determination unit 116 determines that the time of use of the coiled tube has exceeded the upper limit time of use and that the coiled tube cannot be incorporated into a new endoscope. The constituent component determined to be non-reusable is preferably discarded right away. Although the constituent component is preferably discarded immediately, if the constituent component is not to be discarded immediately, the constituent component needs to be managed so as not to be reused. Instead of the image-capturing unit and the coiled tube determined to be non-reusable, a usable image-capturing unit and a usable coiled tube may be assigned to the new endoscope.

The determination unit 116 determines that the constituent component can be incorporated into a new endoscope if the usage count included in the use status of the constituent component is less than the upper limit usage count and if the time of use included in the use status of the constituent component is less than the upper limit time of use (Y in S14). In the record shown in FIG. 7, the usage count and the time of use of each of a bending drum, a bending lever, a connector, and a bending tube are less than the upper limit usage count and the upper limit time of use, respectively. Therefore, the determination unit 116 determines that the bending drum, the bending lever, the connector, and the bending tube that used to be incorporated into the endoscope with scope ID “2001” can be incorporated into a new endoscope, that is, the constituent components are reusable.

The constituent components determined to be reusable are incorporated into a planned endoscope at a manufacturing plant (S18). The constituent components that used to be incorporated into the endoscope with scope ID “2001” may be incorporated into the same endoscope or different endoscopes. The following explains a case in which the bending tube that used to be incorporated in the endoscope with scope ID “2001” is incorporated into a newly manufactured endoscope with scope ID “2915”.

For the endoscope with scope ID “2915”, the recording processing unit 118 records the use status of the components of the endoscope with scope ID “2915” in the recording unit 120. The recording processing unit 118 records the name, the usage count, and the time of use of constituent components of the endoscope in association with scope ID “2915” in the recording unit 120.

More specifically, the recording processing unit 118 extracts the use status of the bending tube from the record containing scope ID “2001” acquired by the second acquisition unit 114 (see FIG. 7). In this example, the use status of the bending tube includes the results of use of two times for the usage count and two hours for the time of use. The recording processing unit 118 records a new endoscope scope ID “2915” in association with the use status of the bending tube (S20).

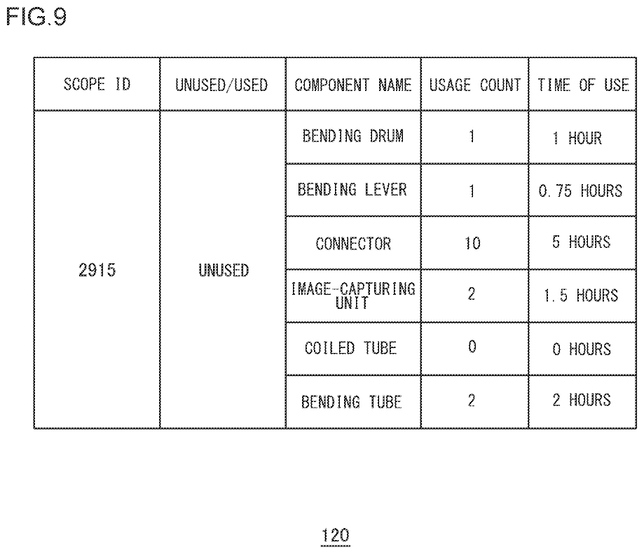

FIG. 9 shows examples of the use status of constituent components of a manufactured endoscope. The recording unit 120 records the name, the usage count, and the time of use of each constituent component of a new endoscope in association with scope ID “2915”.

As described above, the usage count and the time of use recorded in the record for scope ID “2001” (see FIG. 7) are recorded in the fields for the usage count and the time of use of the bending tube. Therefore, the latest use status of the bending tube is recorded in the recording unit 120 in association with scope ID “2915”. With reference to FIG. 9, the endoscope also incorporates the bending drum that used to be incorporated in the endoscope with scope ID “2001”, and the use status of the bending drum reflects the latest use status recorded in the record for scope ID “2001”.

In the endoscope with scope ID “2915”, the bending lever, the connector, and the image-capturing unit are reused after being taken out from an endoscope different from the endoscope with scope ID “2001”. A new component is adopted for the coiled tube. In the first embodiment, the recording unit 120 records the scope ID an endoscope to be newly manufactured in association with the latest use status of each constituent component, thereby allowing the determination unit 116 to easily determine whether or not each constituent component can be reused after the endoscope is used.

### Second Embodiment

In the first embodiment described above, the identification information (component ID) of a constituent component is not used. In the second embodiment, the recording unit 120 records the component ID, the usage count, and the time of use of a constituent component in association with the scope ID of an endoscope.

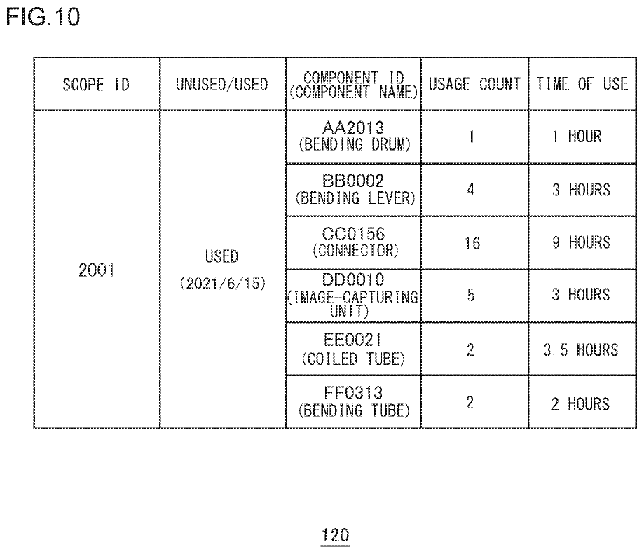

FIG. 10 shows another example of the use status of constituent components of a used endoscope. Compared to the use status shown in FIG. 7, each constituent component is managed by a component ID. A bending drum with component ID “AA2013” has a use status of one for the usage count and one hour for the time of use. The usage count of one means that an endoscope with scope ID “2001” is the only endoscope that has incorporated a bending drum with component ID “AA2013.

A bending tube with component ID “FF0313” has a use status of two for the usage count and two hours for the time of use. The usage count of two means that the bending tube with component ID “FF0313” has been incorporated into two endoscopes and used in two examinations in the past. Therefore, two records of the endoscopes that have incorporated the bending tube with component ID “FF0313” are recorded in the recording unit 120, and the record shown in FIG. 10 is one of the records.

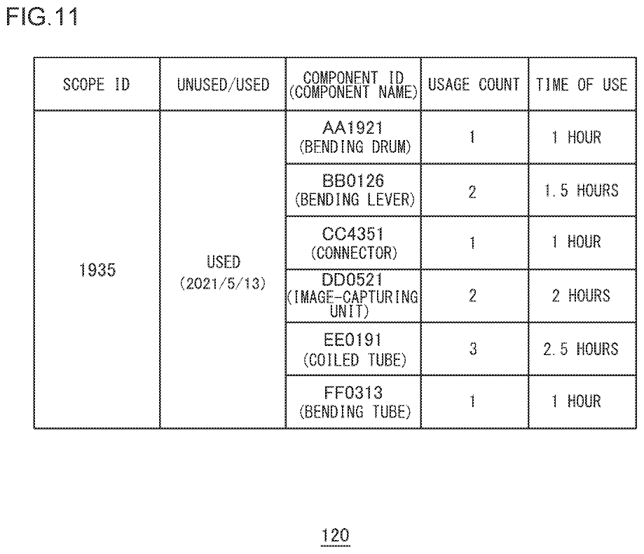

FIG. 11 shows another example of the use status of constituent components of a used endoscope. FIG. 11 shows a record of an endoscope with scope ID “1935” incorporating a bending tube with component ID “FF0313”. In this way, the use status of the bending tube with component ID “FF0313” is recorded in the records shown in FIG. 10 and FIG. 11 in the recording unit 120.

At the time of the manufacturing of a new endoscope, when acquiring the component ID of each constituent component, the recording processing unit 118 searches for the latest record of each constituent component in the recording unit 120. When a bending tube with component ID “FF0313” is incorporated into an endoscope with scope ID “2915”, upon finding multiple records that contain the use status for component ID “FF0313”, the recording processing unit 118 acquires the use status contained in the latest record. Comparing the record shown in FIG. 10 and the record shown in FIF. 11, the record shown in FIG. 10 has a newer use date. Therefore, the recording processing unit 118 may acquire the use status for component ID “FF0313” from the record shown in FIG. 10 and record the use status in association with scope ID “2915” of the endoscope to be newly manufactured.

If the date of use of the records is not used, the recording processing unit 118 may extract the maximum value of the usage count and the maximum value of the time of use from the status for component ID “FF0313” contained in the recording unit 120 as the latest use status. Since the value of the usage count and the value of the time of use are larger for newer records, the recording processing unit 118 may specify the latest use status by extracting the maximum value of the usage count and the maximum value of the time of use.

In a case where constituent components are managed using component IDs, if it is determined that a constituent component is non-reusable (N in S14) in the flowchart shown in FIG. 8, the component ID of the constituent component may be registered and managed as the component ID of a component to be discarded or a component that is not usable. By performing such management, the constituent component determined to be non-reusable is prohibited from being incorporated and reused in a new endoscope.

### Third Embodiment

In the first and second embodiments, examples are shown in which the usage count and the time of use are accumulated and recorded in the recording unit 120 as the status of a constituent component. In the third embodiment, the time used in an examination is recorded as the status of a constituent component, and the accumulated values through multiple examinations are not recorded. An explanation will be given regarding the use status of a bending tube with component ID “FF0313” in the third embodiment. It is assumed that the bending tube with component ID “FF0313” has been used in two examinations in the past.

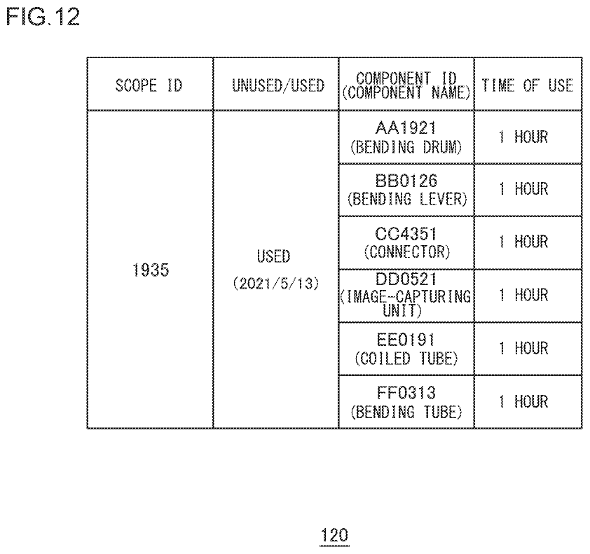

FIG. 12 shows examples of the use status of constituent components of a used endoscope in the third embodiment. FIG. 12 shows a record of an endoscope with scope ID “1935” incorporating a bending tube with component ID “FF0313”. The date of use (examination date) is 2021/5/13. The examination time during which the endoscope was used is one hour. Therefore, one hour of use time is recorded for multiple constituent components including a bending tube.

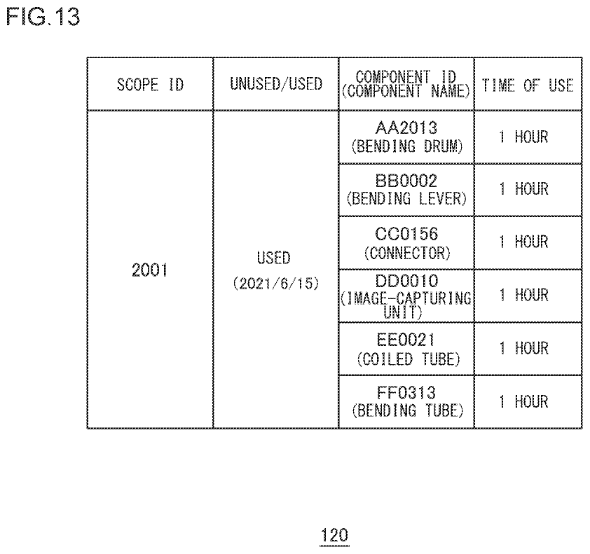

FIG. 13 shows examples of the use status of constituent components of a used endoscope. FIG. 13 shows a record of an endoscope with scope ID “2001” incorporating a bending tube with component ID “FF0313”. The date of use (examination date) is 2021/6/15. The examination time during which the endoscope was used is one hour. Therefore, one hour of use time is recorded for multiple constituent components including a bending tube.

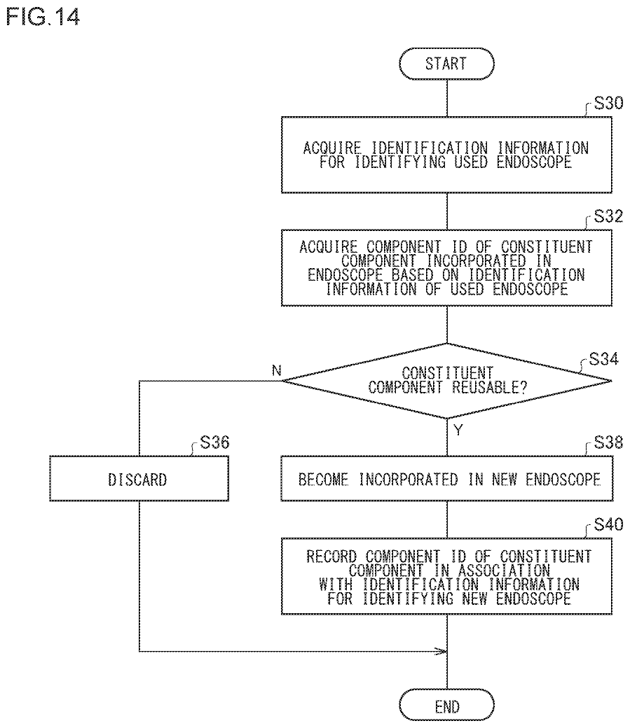

FIG. 14 is a flowchart for managing the status of constituent components. When a used endoscope is brought to a medical device manufacturer, the first acquisition unit 112 acquires identification information for identifying the used endoscope (S30). The first acquisition unit 112 acquires the scope ID of the used endoscope as the identification information. In this case, the first acquisition unit 112 acquires scope ID “2001”.

In the third embodiment, the constituent components that used to be incorporated in scope ID “2001” may be scheduled to be incorporated into a specific new endoscope. The management server 100 recognizes an endoscope in which a constituent component that used to be incorporated in the endoscope with scope ID “2001” is to be newly incorporated. In other words, the management server 100 recognizes the scope ID of an endoscope in which each constituent component to be incorporated into a newly manufactured endoscope was most recently incorporated.

The second acquisition unit 114 acquires identification information (component ID) for identifying a constituent component to be incorporated into a new endoscope based on scope ID “2001” of the endoscope acquired by the first acquisition unit 112 (S32). More specifically, the second acquisition unit 114 refers to the recording unit 120 and acquires the component ID of the constituent component scheduled to be incorporated into the new endoscope from a record including scope ID “2001” (see FIG. 13). In this example, the second acquisition unit 114 acquires the component ID “FF0313”.

The determination unit 116 determines whether or not the constituent component can be incorporated into the new endoscope based on the component ID of the constituent component acquired by the second acquisition unit 114 (S34). More specifically, the determination unit 116 identifies records that include the component ID in the recording unit 120 and derives the usage count and the time of use of the constituent component based on one or more identified records. At this time, the determination unit 116 may use the number of the identified records as the usage count of the constituent component. The determination unit 116 may also derive the time of use of the constituent component by summing up the time of use included in each record.

The determination unit 116 compares the derived usage count and time of use of the constituent component with the upper limit usage count and the upper limit time of use held in the upper limit holding unit 122. The determination unit 116 determines that the constituent component cannot be incorporated into a new endoscope if the derived usage count is equal to or greater than the upper limit usage count and determines that the constituent component cannot be incorporated into a new endoscope if the derived time of use is equal to or greater than the upper limit time of use (N in S34). The constituent component determined to be non-reusable may be discarded (S36).

The determination unit 116 determines that the constituent component can be incorporated into a new endoscope if the derived usage count is less than the upper limit usage count and if the derived time of use is less than the upper limit time of use (Y in S34). Based on the records shown in FIG. 12 and FIG. 13, the usage count and time of use of the bending tube with component ID “FF0313” are derived to be two and two hours, respectively. The derived usage count and the derived time of use are less than the upper limit usage count and the upper limit time of use that are set for the bending tube. Therefore, the determination unit 116 determines that the bending tube with component ID “FF0313” can be incorporated into a new endoscope, that is, the bending tube is reusable.

The constituent component determined to be reusable is incorporated into a planned endoscope at a manufacturing plant (S38). The following explains a case in which the bending tube with component ID “FF0313” that used to be incorporated in the endoscope with scope ID “2001” is incorporated into a newly manufactured endoscope with scope ID “2915”.

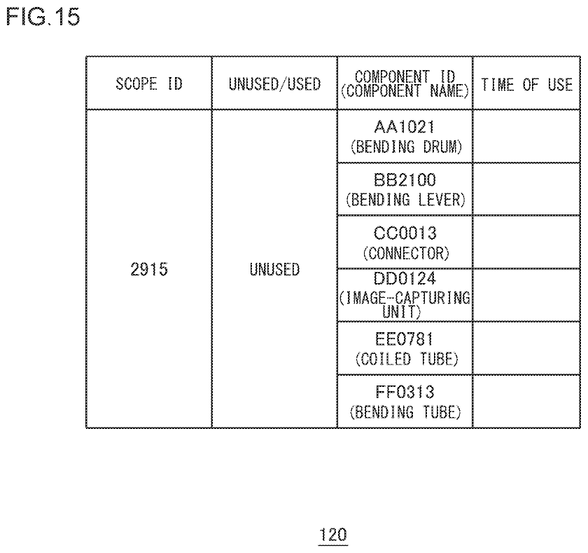

For the endoscope with scope ID “2915”, the recording processing unit 118 creates a record of the endoscope with scope ID “2915” in the recording unit 120. FIG. 15 shows examples of the records of constituent components of a manufactured endoscope. The recording unit 120 records the component IDs of constituent components of a new endoscope in association with scope ID “2915” (S40). In the third embodiment, the field for the time of use is blank when the endoscope with scope ID “2915” has not been used, and the recording processing unit 118 records the time of use once the endoscope is used. In the third embodiment, the recording unit 120 records a record in which the component ID of each constituent component is associated with the scope ID of an endoscope to be newly manufactured, and only the time of use is recorded after the endoscope is used. Therefore, the record management in the recording unit 120 can be easily realized.

### Fourth Embodiment

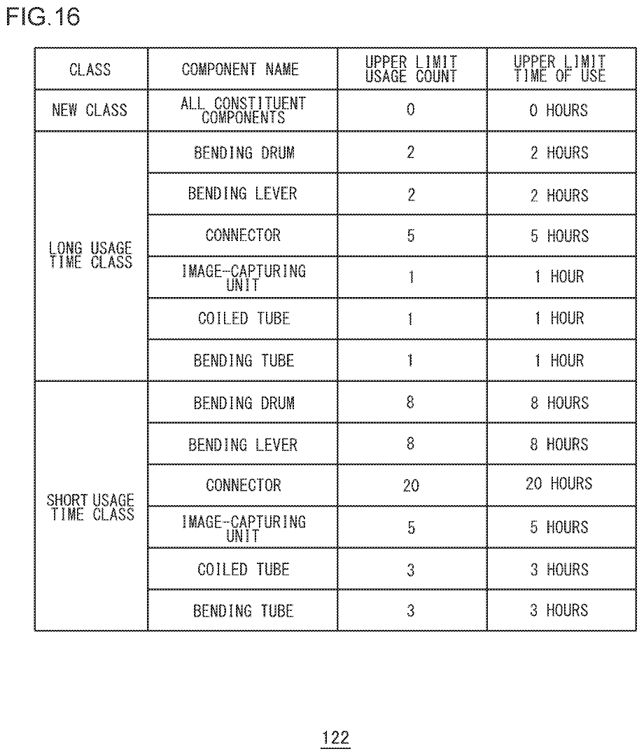

In the fourth embodiment, classes are set for endoscopes to be remanufactured, and the endoscopes are classified according to the use status of the constituent components. FIG. 16 shows examples of upper limit usage counts and upper limit times of use of constituent components that are set for a plurality of classes of a single-use endoscope. FIG. 16 representatively shows the upper limit usage counts and the upper limit times of use for six components constituting the single-use endoscope. Upper limit usage counts and upper limit times of use for other components may also be set for each endoscope class.

<New Class>

A new class is a class in which new constituent components are employed for all the constituent components. Therefore, the upper limit usage count and the upper limit time of use of the constituent components to be incorporated are set to zero.

<Long Usage Time Class>

A long usage time class is a class that guarantees the use for a long examination. Therefore, the upper limit usage count and the upper limit time of use of the constituent components to be incorporated are set to have relatively small values.

<Short Usage Time Class>

A short usage time class is a class that guarantees the use for a short examination. Therefore, the upper limit usage count and the upper limit time of use of the constituent components to be incorporated may be set to have larger values compared to those in the long usage time class.

The upper limit holding unit 122 holds the upper limit usage count and the upper limit time of use for each constituent component for each class. In the fourth embodiment, the upper limit holding unit 122 holds the upper limit usage count and the upper limit time of use. Alternatively, the upper limit holding unit 122 may hold either one of the upper limit usage count and the upper limit time of use. The upper limit usage count and the upper limit time of use may be set by the medical device manufacturer in consideration of safety. The selling price, lease fee, contract fee, etc., of an endoscope may be determined according to the class and may be set higher in the order of the new class, the long usage time class, and the short usage time class.

When remanufacturing an endoscope of a certain class, the determination unit 116 determines whether or not a component scheduled for remanufacturing of the endoscope can be used based on the upper limit usage count and upper limit time of use for each constituent component set for that class. For example, when manufacturing an endoscope of the long usage time class, the determination unit 116 determines that those that can be used for the endoscope of the long usage time class are only components with a use status of less than the upper limit usage count and less than the upper limit time of use that are set for each constituent component of the long usage time class. By setting endoscope class in this way, it is possible to use an endoscope that matches the patient's wishes for the examination.

Based on the use status of constituent components included in a newly manufactured endoscope, the classification unit 126 may determine the class of the endoscope. More specifically, the classification unit 126 classifies the endoscope into a class in which the respective use statuses of all the constituent components satisfy the condition and that has a lower reuse rate. The reuse rate is lower in the order of the new class, the long usage time class, and the short usage time class. The following is an explanation regarding the endoscope records shown in FIG. 9.

In the record shown in FIG. 9, the coiled tube is new and meets all the conditions of the new class, the long usage time class, and the short usage time class. Next, the use status of the curved drum and the use status of curved lever satisfy the conditions of the long usage time class and the short usage time class. The respective use statuses of the connector, the image-capturing unit, and the bending tube satisfy only the condition of the short usage time class. As described, since the respective use statuses of all the constituent components satisfy the condition of the short usage time class, the classification unit 126 classifies the endoscope with scope ID “2915” shown in FIG. 9 into the short usage time class. By classifying endoscopes after remanufacturing in this way, it is possible to use an endoscope that matches the patient's wishes for the examination.

### Fifth Embodiment

Single-use endoscopes are transported to medical facilities while being packed in sterile packs, and after being used at the medical facilities, the endoscopes are transported from the medical facilities to the medical device manufacturer. Since there are cases where the strength and durability of components constituting single-use endoscopes are intentionally set to be low, the quality may deteriorate if the number of times of transportation is large or the transportation time is long. Therefore, in the fifth embodiment, the recording unit 120 records the results of transportation of constituent components and manages the transportation status of the constituent components.

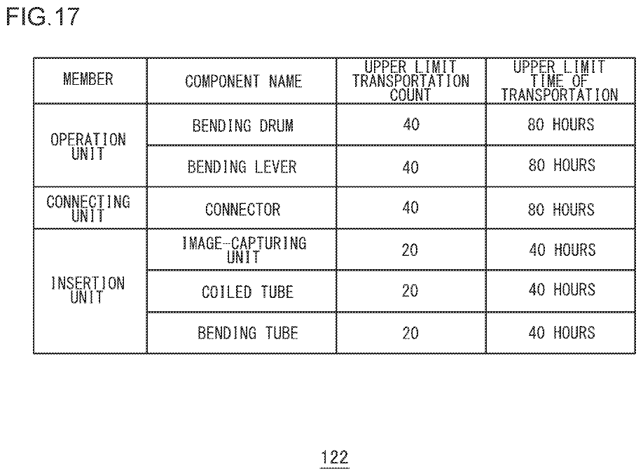

FIG. 17 shows examples of upper limit transportation counts and upper limit times of transportation that are set for constituent components of a single-use endoscope. FIG. 17 representatively shows the upper limit transportation counts and the upper limit times of transportation for six components constituting the single-use endoscope. Upper limit transportation counts and upper limit times of transportation for other components may also be set.

The upper limit holding unit 122 holds the upper limit transportation count and the upper limit time of transportation for each constituent component. The upper limit transportation count is the maximum number of times a constituent component is transported, and the upper limit time of transportation is the maximum total amount of time spent for transportation. When the results of transportation of the constituent component exceeds either the upper limit transportation count or the upper limit time of transportation, the constituent component may be discarded. In the embodiment, the upper limit holding unit 122 holds the upper limit transportation count and the upper limit time of transportation. Alternatively, the upper limit holding unit 122 may hold either one of the upper limit transportation count and the upper limit time of transportation. The upper limit transportation count and the upper limit time of transportation may be set by the medical device manufacturer in consideration of safety.

In the fifth embodiment, the recording unit 120 records the results of transportation of constituent components and manages the transportation status of the constituent components. In association with identification information (scope ID) of a manufactured endoscope, the recording unit 120 according to the fifth embodiment may record the accumulated values of the transportation count and/or the time of transportation for a constituent component constituting the endoscope. Endoscopes transported from a manufacturing facility to a medical facility are transported from the medical facility to the manufacturing facility after being used. The recording processing unit 118 may derive the time of transportation spent for the transportation of an endoscope based on the transportation route of the endoscope and record the time of transportation in the recording unit 120. By installing a GPS receiver or various sensors in a container box that accommodates the endoscope to be transported, the time of transportation may be derived and recorded in the recording unit 120 by the recording processing unit 118.

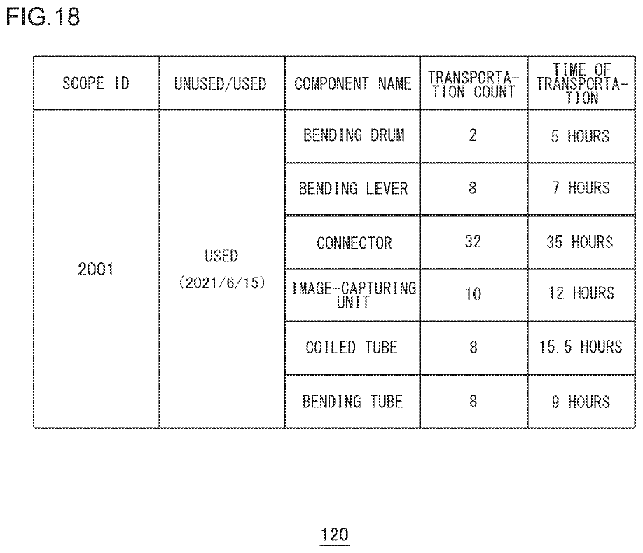

FIG. 18 shows examples of the transportation status of constituent components of a used endoscope. When remanufacturing an endoscope, the determination unit 116 determines whether or not a component scheduled for the remanufacturing can be used based on the upper limit transportation count and upper limit time of transportation of constituent components. More specifically, the determination unit 116 compares the transportation count and the time of transportation of the constituent component recorded in the recording unit 120 with the upper limit transportation count and the upper limit time of transportation held in the upper limit holding unit 122. The determination unit 116 determines that the constituent component cannot be incorporated into a new endoscope if the transportation count included in the transportation status of the constituent component is equal to or greater than the upper limit transportation count and determines that the constituent component cannot be incorporated into a new endoscope if the time of transportation included in the transportation status of the constituent component is equal to or greater than the upper limit time of transportation.

On the other hand, the determination unit 116 may determine that the constituent component can be incorporated into a new endoscope if the transportation count included in the transportation status of the constituent component is less than the upper limit transportation count and if the time of transportation included in the transportation status of the constituent component is less than the upper limit time of transportation.

Described above is an explanation on the present disclosure based on the plurality of embodiments. These embodiments are intended to be illustrative only, and it will be obvious to those skilled in the art that the embodiments may be combined, that various modifications to constituting elements and processes could be developed, and that further modifications are also within the scope of the present disclosure.

For example, in the first embodiment, the recording unit 120 records the respective accumulated values of a usage count and a time of use as a use status, and the determination unit 116 performs a comparison with an upper limit usage count and an upper limit time of use. In an exemplary variation, the recording unit 120 may record a remaining usage count and a remaining time of use as a use status, and the determination unit 116 may compare the remaining usage count and the remaining time of use with zero, which is a threshold value. The recording processing unit 118 may derive the remaining usage count as “(upper limit usage count−usage count)”, and the remaining time of use may be derived as “(upper limit time of use−time of use)”.

In each embodiment, the management server 100 is provided in the external system 3 outside the medical facility. Alternatively, the management server 100 may be provided in the facility system 2 and communicably connected to the facility management unit 20 via the network 22.

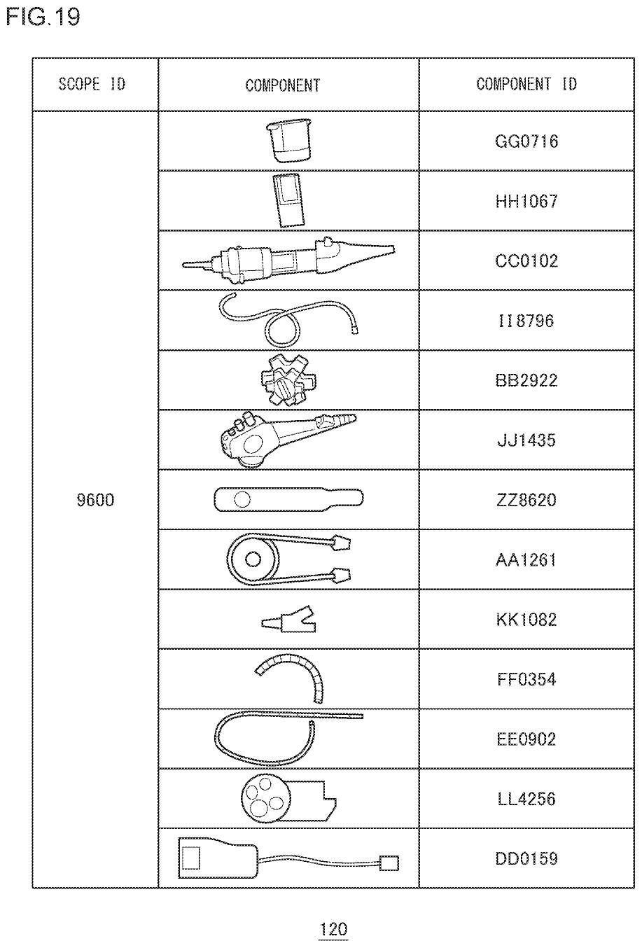

In each embodiment, when a used endoscope is collected, the first acquisition unit 112 acquires a scope ID assigned to the endoscope as identification information for identifying the used endoscope (S10, S30). In the second embodiment, component IDs of a plurality of components constituting an endoscope are managed in association with the scope ID. Therefore, by the acquisition of the scope ID by the first acquisition unit 112, the second acquisition unit 114 acquires the use status of constituent components to be incorporated into a new endoscope based on the scope ID. FIG. 19 shows an example of a state where component IDs of a plurality of constituent components are associated with a scope ID. As described, in the embodiment, since constituent components are associated with a scope ID, the first acquisition unit 112 acquires the scope ID as identification information for identifying a used endoscope.

In the exemplary variation, the first acquisition unit 112 acquires identification information of a specific component incorporated in a used endoscope as identification information for identifying the used endoscope. In this exemplary variation, the identification information of the specific component is used as identification information for identifying an endoscope. For example, the specific component may be a key component of the endoscope, which may be, for example, the main frame of the operating unit (a plate or cylindrical member in which various components and operating devices are mounted that are referred to as baseplates, or the like), the main unit of a channel branching component, or the main component of the connecting unit (for example, a circuit component that carries an RF tag and transmits and receives signals from the control device 10 or the main frame of a connector housing). In other words, in the exemplary variation, a component ID assigned to one component that constitutes an endoscope may be used as identification information for identifying the endoscope.

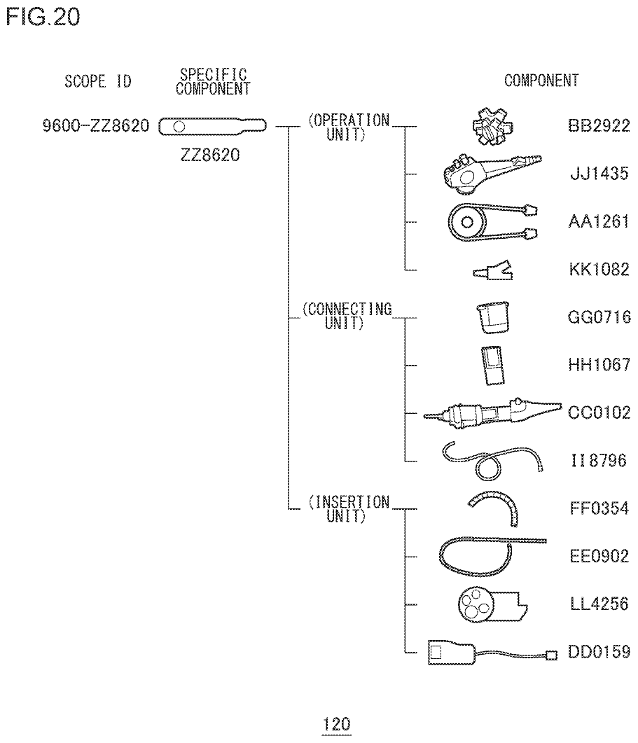

FIG. 20 shows an example of a state where component IDs of a plurality of constituent components of an endoscope are associated with a component ID of a specific constituent component. In this example, the component ID of the main frame of an operating unit is used as identification information for identifying the endoscope, and the component IDs of a plurality of constituent components constituting the endoscope are associated with the component ID of the operating unit main frame. Among the plurality of components, a component representing the endoscope may be the main component of the connecting unit.

When a key component is discarded, the recording processing unit 118 preferably holds a record for the key component in the recording unit 120 until all the constituent components whose use status is being managed in association with the component ID of the key component are discarded.

The usage counts and times of use of constituent components may be used as materials for analysis when an abnormality occurs in the endoscope. When an abnormality occurs in the endoscope, an appropriate upper limit usage count and an upper limit time of use may be re-set by analyzing the usage count and time of use of the constituent components.

In the embodiments, the management server 100 holds the second acquisition unit 114 and the recording unit 120. Alternatively, a structure may be provided in an endoscope that acquires the status of components constituting the endoscope and/or the component IDs of the components constituting the endoscope and records the status and/or the component IDs in association with the identification information of the endoscope. In other words, the endoscope may have the functions of the second acquisition unit 114 and the recording unit 120, which are some of the functions of the management server 100.

What is claimed is:

1. A medical device management system comprising:
a first acquisition unit that acquires first identification information for identifying a first medical device having a first constituent component and a second constituent component;
a second acquisition unit that acquires a status of the first constituent component incorporated in a second medical device or second identification information for identifying the first constituent component incorporated in the second medical device based on the first identification information; and
a recording unit that records the status of the first constituent component or the second identification information and third identification information for identifying the second medical device in association with each other.

2. The medical device management system according to claim 1, wherein
the first medical device is a single-use medical device, and
the status of the first constituent component includes the number of times a medical device or medical devices incorporating the first constituent component were used in a medical practice.

3. The medical device management system according to claim 1, wherein
the status of the first constituent component includes total amount of time during which a medical device or medical devices incorporating the first constituent component were used in a medical practice.

4. The medical device management system according to claim 1, wherein
the status of the first constituent component includes the number of times a medical device or medical devices incorporating the first constituent component were transported or total amount of time for which the medical device or medical devices incorporating the first constituent component were transported.

5. The medical device management system according to claim 1, wherein
the first identification information is identification information assigned to the first medical device.

6. The medical device management system according to claim 1, wherein
the second constituent component is a key component of the first medical device, and
the first identification information is identification information assigned to the second constituent component.

7. The medical device management system according to claim 1, wherein
the recording unit that is provided in a management server that manages the first medical device and the second medical device.

8. The medical device management system according to claim 1, wherein
the recording unit is provided in the second medical device.

9. The medical device management system according to claim 1, further comprising:
a determination unit that determines whether or not the first constituent component can be incorporated into a new medical device based on the status of the first constituent component, wherein
the determination unit determines that the first constituent component cannot be incorporated into the new medical device if the number of times used in a medical practice included in the status of the first constituent component is equal to or more than an upper limit usage count.

10. The medical device management system according to claim 1, further comprising:
a determination unit that determines whether or not the first constituent component can be incorporated into a new medical device based on the second identification information, wherein
the determination unit derives the number of times the first constituent component has been used in a medical practice from a record associated with the second identification information in the recording unit and determines that the first constituent component cannot be incorporated into the new medical device if the number of times the first constituent component has been used is equal to or more than an upper limit usage count.

11. The medical device management system according to claim 1, comprising:
a classification unit that determines, based on the status of a constituent component included in the second medical device manufactured, the class of the medical device.

12. The medical device management system according to claim 1, wherein
the first medical device and the second medical device are endoscopes, and
the first constituent component is an image-capturing unit of an endoscope.

13. The medical device management system according to claim 1, wherein
the first medical device and the second medical device are endoscopes, and
the second constituent component is a main frame of an operating unit of an endoscope.

14. The medical device management system according to claim 1, wherein
the first medical device and the second medical device are endoscopes, and
the second constituent component is a main component of a connecting unit of an endoscope.

15. A management server that manages a medical device, comprising:
a first acquisition unit that acquires first identification information for identifying a first medical device having a first constituent component and a second constituent component;
a second acquisition unit that acquires a status of the first constituent component incorporated in a second medical device or second identification information for identifying the first constituent component incorporated in the second medical device based on the first identification information; and
a recording unit that records the status of the first constituent component or the second identification information and third identification information for identifying the second medical device in association with each other.

16. A medical device having a plurality of constituent components, comprising:
an acquisition unit that acquires, based on first identification information for identifying another medical device that used to incorporate the constituent component, a status of the constituent component that used to be incorporated in the another medical device or second identification information for identifying the constituent component that used to be incorporated in the another medical device; and
a recording unit that records the status of the constituent component or the second identification information and third identification information for identifying the medical device in association with each other.

17. A medical device management method comprising:
acquiring first identification information for identifying a first medical device having a first constituent component and a second constituent component;
acquiring a status of the first constituent component incorporated in a second medical device or second identification information for identifying the first constituent component incorporated in the second medical device based on the first identification information; and
recording the status of the first constituent component or the second identification information and third identification information for identifying the second medical device in association with each other.
